(19) United States
(12) Patent Application Publication
(10) Pub. No.: US 2016/0045418 A1
(43) Pub. Date: Feb. 18, 2016

(54) HAIR CARE SHAMPOO

(71) Applicant: Henkel AG & Co. KGaA, Duesseldorf (DE)

(72) Inventors: Thomas Schroeder, Hamburg (DE); Dirk Hentrich, Hamburg (DE); Marine Wassen, Luxemburg (LU)

(21) Appl. No.: 14/923,742

(22) Filed: Oct. 27, 2015

Related U.S. Application Data
(63) Continuation of application No. PCT/EP2014/052048, filed on Feb. 3, 2014.

(30) Foreign Application Priority Data
May 2, 2013 (DE) 10 2013 208 056.5

(51) Int. Cl. A61K 8/46; A61K 8/92; A61Q 5/02; A61K 8/37; A61K 8/73

(52) U.S. Cl. CPC A61K 8/463; A61K 8/375; A61K 8/737; A61Q 5/02; A61K 8/922

(57) ABSTRACT

A cleaning cosmetic composition includes, in a cosmetic carrier, a) at least one anionic surfactant (A), b) optionally, an amphoteric surfactant (B), c) at least one sorbitan acid ester (C), and d) at least one cationic polysaccharide (D).

### FIELD OF THE INVENTION

The present invention generally relates to a cleaning cosmetic composition suitable, for example, as a hair shampoo for the cleaning of hair.

### BACKGROUND OF THE INVENTION

Cosmetic cleaning agents such as, for example, hair shampoos, are based on classic anionic, amphoteric, zwitterionic, nonionic, and/or cationic surfactants, whereby because of their excellent cleaning and foaming ability, predominantly anionic surfactants are typically used, optionally mixed with small amounts of co-surfactants. Suitable commercial shampoos clean the hair and remove sebaceous residues and/or residues of styling agents and other impurities from the hair surface and the scalp.

However, lipids and proteins are also removed from the hair and scalp during the cleaning, which can damage the hair structure and cause the scalp to dry out, particularly with frequent cleaning. To eliminate these disadvantages, therefore surfactants that are gentle and well tolerated by the hair and scalp have been used preferably in hair cleaning agents in recent years.

Gentle surfactants are in fact known per se, but these do not yet satisfy all consumer requirements to a sufficient extent. Thus, it has become evident, for example, that some formulations based on gentle surfactants cannot always be produced with the desired textures. To achieve the desired texture, i.e., to establish the desired “creamy” flow behavior and a higher viscosity of the agents, extensive formulation work is necessary and the long-term stability of these agents is often not satisfactory. Furthermore, gentle surfactants are often not highly foaming and reduce the amount and quality of the foam (creaminess and fine-pored nature of the foam). The incorporation of care substances into hair cleaning agents can enhance this effect further.

Gentle surfactants are in fact known per se, but these do not yet satisfy all consumer requirements to a sufficient extent. Thus, it has become evident, for example, that some formulations based on gentle surfactants cannot always be produced with the desired textures. To achieve the desired texture, i.e., to establish the desired “creamy” flow behavior and a higher viscosity of the agents, extensive formulation work is necessary and the long-term stability of these agents is often not satisfactory. Furthermore, gentle surfactants are often not highly foaming and reduce the amount and quality of the foam (creaminess and fine-pored nature of the foam). The incorporation of care substances into hair cleaning agents can enhance this effect further.

It is therefore desirable to provide gentle, skin- and hair-compatible, and caring hair cleaning agents with an improved texture and foam quality. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It has now been found that this object is achieved by a specific active ingredient combination of the anionic surfactant sorbitan sesquioctanoate and a cationic polysaccharide. The corresponding cleaning agents are skin- and hair-compatible, care for and clean the hair, and can be easily distributed on the hair because of their advantageous rheological properties, and in combination with water form a creamy, fine-pored foam.

Such a cleaning cosmetic composition includes, in a cosmetic carrier, at least one anionic surfactant (A), optionally an amphoteric surfactant (B), at least one sorbitan acid ester (C), at least one cationic polysaccharide (D).

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

A first subject of the invention is a cleaning cosmetic composition, which includes, in a cosmetic carrier,
a) at least one anionic surfactant (A),
b) optionally an amphoteric surfactant (B),
c) at least one sorbitan acid ester (C),
d) at least one cationic polysaccharide (D).

The cleaning cosmetics according to the invention include as a first essential ingredient at least one anionic surfactant (A), preferably being used in only relatively small amounts. It is preferred that the percentage by weight of the anionic surfactant (A) relative to the total weight of the composition is 6.0 to 15.0% by weight, preferably 8.0 to 11.0% by weight.

Suitable anionic surfactants (A), which may be used in the agents according to the invention, include:
linear and branched fatty acids having 8 to 30 C atoms (soaps),
ether carboxylic acids of the formula R—O—(CH₂—CH₂O)ₓ—CH₂—COOH, in which R is a linear or branched, saturated or unsaturated alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and/or dialkyl esters having 8 to 24 C atoms in the alkyl group and sulfosuccinic acid monoalkylpolyoxyethyl esters having 8 to 24 C atoms in the alkyl group and 1 to 6 oxyethyl groups,
alpha-olefinsulfonates having 8 to 24 C atoms,
alkyl sulfate and/or alkyl polyglycol ether sulfate salts of the formula R—(OCH₂—CH₂)ₓ—OSO₃⁻X⁺, in which R is a preferably linear or branched, saturated or unsaturated alkyl group having 8 to 30 C atoms, x=0 or 1 to 12, and X an alkali or ammonium ion,
sulfonates of unsaturated fatty acids having 8 to 24 C atoms and 1 to 6 double bonds,
esters of tartaric acid and citric acid with alcohols, which are adducts of about 2-15 molecules of ethylene oxide and/or propylene oxide to fatty alcohols having 8 to 22 C atoms,
alkyl and/or alkenyl ether phosphates of the formula,

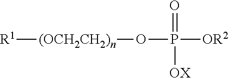

in which R¹ preferably stands for an aliphatic hydrocarbon group having 8 to 30 carbon atoms, R² for hydrogen, a group (CH₂CH₂O)ₙR¹ or X, n for numbers from 0 to 10, and X for hydrogen, an alkali metal or alkaline earth metal, or NR³R⁴R⁵R⁶, where R³ to R⁶ independently of one another stand for a C₁ to C₄ hydrocarbon group.

Preferred anionic surfactants are ether carboxylic acids of the aforementioned formula, acyl sarcosides having 8 to 24 C atoms in the acyl group, sulfosuccinic acid mono- and/or -dialkyl esters having 8 to 24 C atoms in the alkyl group and sulfosuccinic acid monoalkylpolyoxyethyl esters having 8 to 24 C atoms in the alkyl group and 1 to 6 oxyethyl groups, alpha-olefinsulfonates having 8 to 24 C atoms, and/or alkyl sulfate salts and/or alkyl polyglycol ether sulfate salts of the aforementioned formula.

Especially preferred anionic surfactants are straight-chain or branched alkyl ether sulfates, which include an alkyl group having 8 to 18 and especially having 10 to 16 C atoms, and 1 to 6 and particularly 2 to 4 ethylene oxide units. Furthermore, especially preferred anionic surfactants are straight-chain or branched alkyl sulfonates, which include an alkyl group having 8 to 18 and especially having 10 to 16 C atoms. Preferred in particular are the sodium, magnesium, and/or triethanolamine salts of linear or branched lauryl, tridecyl, and/or myristyl sulfates, which have a degree of ethoxylation of 2 to 4.

Cosmetics very especially preferred according to the invention are characterized in that they include at least one anionic surfactant (A) from the group of alkyl sulfates and alkyl polyglycol ether sulfates of the formula R—O—(CH₂—CH₂O)ₙ—O—SO₃X, in which R preferably stands for a straight-chain or branched, saturated or mono- or polyunsaturated alkyl or alkenyl group having 8 to 24 carbon atoms, n for 0 or 1 to 12, and X for an alkali metal or alkaline earth metal or for triethanolamine, whereby anionic surfactants with the INCI name Sodium Lauryl Sulfate are especially preferred.

The cosmetics according to the invention include at least one amphoteric surfactant (B) as a second essential ingredient. Amphoteric surfactants are added to the agents according to the invention, based on their total weight, preferably in amounts from 1.0 to 12% by weight, more preferably from 2.0 to 10% by weight, and especially from 3.0 to 6.0% by weight. Suitable amphoteric surfactants can be selected from compounds of the following formulas (i) to (v), in which the group R in each case stands for a straight-chain or branched, saturated or mono- or polyunsaturated alkyl or alkenyl group having 8 to 24 carbon atoms,

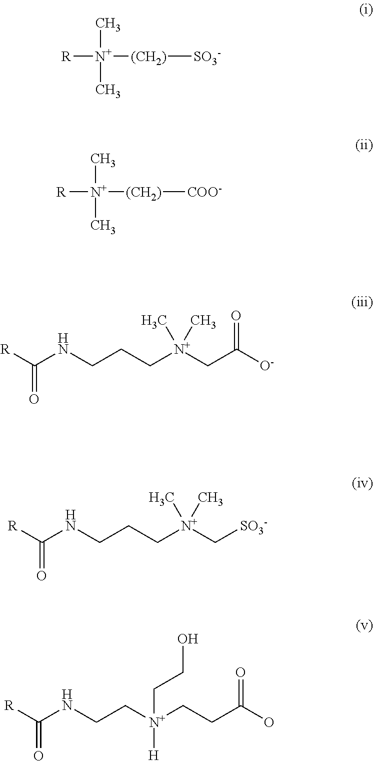

Especially suitable amphoteric surfactants are alkyl amidoalkyl betaines and/or alkyl ampho(di)acetates of the aforementioned formulas (i) to (v). Especially suitable amphoteric surfactants include the surfactants known under the INCI names Cocamidopropyl Betaine and Disodium Cocoamphodiacetate. Cosmetics especially preferred according to the invention are characterized in that they include at least one amphoteric surfactant (B) from the group of the general formula (iii), whereby amphoteric surfactants with the INCI name Cocamidopropyl Betaine are especially preferred.

Preferred compositions according to the invention are characterized in that the percentage by weight of the anionic surfactant (A) and amphoteric surfactant (B) in the composition, based on the total weight of the composition, is 6.0 to 15% by weight, preferably 8.0 to 11% by weight.

The third essential ingredient of the cosmetic compositions according to the invention is at least one sorbitan acid ester (C). The percentage by weight of the sorbitan acid ester relative to the total weight of the composition is preferably 0.1 to 3.0% by weight, preferably 0.25 to 1.0% by weight.

Suitable sorbitan acid esters (C), which may be used in the cleaning agents according to the invention, are understood preferably as esters of sorbitan with straight-chain or branched, saturated or unsaturated carboxylic acids having 4 to 16, preferably having 5 to 12, and especially having 6 to 10 carbon atoms in the molecule.

Sorbitans are derived from the sugar alcohol sorbitol, which forms cyclic internal ethers with the elimination of water. Sorbitans typically represent a product mixture, which consists predominantly of ethers of the following formulas (III) to (V):

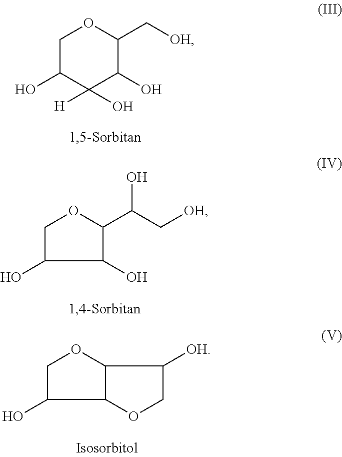 [SMILES: OCC1OCC(O)C(O)C1O ; OCC(O)C1OCC(O)C1O ; OC1COC2C(O)COC12]

Sorbitans include in minor amounts other condensation products as well and sorbitol.

Sorbitan esters are accordingly understood preferably as mixtures of the aforementioned polyol mixtures with the aforementioned carboxylic acids, whereby it is preferred furthermore if one mole of the polyol mixture is esterified with 1 to 3 mol of carboxylic acid.

Compositions especially preferred according to the invention include the above-described sorbitan esters (C), which are formed by the esterification of the sorbitan with caproic acid, oenanthic acid, caprylic acid, pelargonic acid, and/or capric acid. Especially preferred are sorbitan esters formed from sorbitan and caprylic acid.

Further especially preferred cleaning agents include the above-described sorbitan carboxylic acid esters, which preferably have a hydroxyl number of more than 350, more preferably of more than 400, and especially preferably of more than 450. The hydroxyl number can be determined by conventional determination methods known in the existing art, for example, according to DGF C-V 17a (53), Ph. Eur. 2.5.3 Method A and DIN 53240.

Especially preferred compositions include at least one sorbitan acid ester (C) with the INCI name Sorbitan Sesquicaprylate.

A last essential ingredient of the cosmetic compositions of the invention is the cationic polysaccharide (D), whose percentage by weight relative to the total weight of the composition is preferably 0.01 to 1.0% by weight, preferably from 0.1 to 0.5% by weight.

Cationic polysaccharides (E) preferred according to the invention are
quaternized cellulose derivatives, as are commercially available under the names Celquat® and Polymer JR®. The compounds Celquat® H 100, Celquat® L 200, and Polymer JR®400 are preferred quaternized cellulose derivatives,
hydrophobically modified cellulose derivatives, for example, the cationic polymers sold under the trade name SoftCat®,
cationic alkyl polyglycosides,
cationized honey, for example, the commercial product Honeyquat® 50,
cationic guar derivatives, such as in particular the products sold under the trade names Cosmedia® Guar and Jaguar®.

In a preferred embodiment of the invention, the compositions include furthermore at least one aforementioned cationic quaternized cellulose polymer and/or a cationic guar derivative. Preferred are the quaternized cellulose polymers known under the INCI name Polyquatemium-10 (Polymer JR® or Celquat® polymers).

Especially preferred cationic polysaccharides (E) are the cationic hydroxyalkyl-guar derivatives, preferably cationic hydroxyethyltrimethylammonium guar and/or cationic hydroxypropyltrimethylammonium guar with average molecular weights between 800,000 and 1,600,000 daltons. Especially preferred are cationic guar polymers, known under the INCI name Guar Hydroxypropyltrimonium Chloride, with a molecular weight (weight average) between 800,000 and 1,600,000 daltons. The cationic charge density of said guar polymers is preferably at least 0.5 mEq/g, preferably at least 0.6 mEq/g, and especially at least 0.8 mEq/g. Their nitrogen content is preferably within the range from 1.1 to 1.6% by weight (based on their total weight). In summary, cosmetic compositions according to the invention are preferred which include a cationic guar derivative as the cationic polysaccharide (E).

A composition especially advantageous with respect to its cosmetic action includes in a cosmetic carrier, based on its total weight,
a) anionic surfactant with the INCI name Sodium Lauryl Sulfate,
b) optionally an amphoteric surfactant (B),
c) 0.1 to 3.0% by weight of sorbitan sesquioctanoate,
d) 0.01 to 1.0% by weight of cationic polysaccharide (D),
   whereby the percentage by weight of the anionic surfactant (A) in the composition and the optionally contained amphoteric surfactant (B) is 6.0 to 15% by weight, preferably 8.0 to 11% by weight.

The compositions according to the invention include components a) to f) in a cosmetic carrier, which may be preferably aqueous or aqueous-alcoholic. The cosmetic carrier preferably includes at least 40% by weight, preferably 60 to 95% by weight, and especially 75 to 90% by weight of water.

Further, the cosmetic carrier can include at least one alcohol, which can be selected from ethanol, ethyl diglycol, 1-propanol, 2-propanol, isopropanol, 1,2-propylene glycol, glycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, sorbitol, benzyl alcohol, phenoxyethanol, or mixtures of said alcohols. Water-soluble alcohols are preferred. Preferred in particular are ethanol, ethyl diglycol, 1-propanol, 2-propanol, isopropanol, 1,2-propylene glycol, glycerol, benzyl alcohol, and/or phenoxyethanol, as well as mixtures of said alcohols.

Apart from the aforementioned active ingredients and carrier substances, the cosmetic compositions according to the invention can include a number of other ingredients, which give them advantageous properties. The preferred optional active substances, which may be used in the compositions according to the invention, include, for example:
nonionic surfactants and/or nonionic emulsifiers, which may be used in the cosmetic compositions (based on their total weight) preferably in an amount from 0.1 to 7.5% by weight, preferably 0.25 to 5% by weight, and especially 0.5 to 3% by weight.
further oil, wax, and/or fat components, which may be used in the cosmetic compositions (based on their total weight) preferably in an amount from 0.01 to 20% by weight, especially preferably from 0.05 to 15% by weight, and especially from 0.1 to 10% by weight,
antidandruff active agents, which may be used in the cosmetic compositions (based on their total weight) preferably in an amount from 0.025 to 7.5% by weight, particularly preferably from 0.05 to 5% by weight, and especially from 0.075 to 3% by weight.

Suitable nonionic surfactants/emulsifiers include, for example,
C₈-C₃₀ fatty acid monoesters and diesters of adducts of 1 to 30 mol of ethylene oxide to glycerol,
amine oxides,
adducts of 2 to 50 mol of ethylene oxide and/or 0 to 5 mol of propylene oxide to linear and branched fatty alcohols having 8 to 30 C atoms, to fatty acids having 8 to 30 C atoms, and to alkyl phenols having 8 to 15 C atoms in the alkyl group,
sorbitan fatty acid esters and adducts of ethylene oxide to sorbitan fatty acid esters such as, for example, polysorbates,
sugar fatty acid esters and adducts of ethylene oxide to sugar fatty acid esters,
fatty acid alkanolamides,
adducts of ethylene oxide to fatty amines, and/or
alkyl polyglucosides.

Especially suitable nonionic surfactants are alkyl oligoglucosides, especially alkyl oligoglucosides based on hydrogenated C_{12/14} coconut alcohol with a DP of 1 to 3, as they are commercially available, for example, under the INCI name “Coco-Glucosides,” and fatty acid alkanolamides, especially C₈-C₂₄ fatty acid (di)ethanolamides.

Preferred nonionic surfactants furthermore are the C₈-C₃₀ fatty acid monoesters and diesters of adducts of 1 to 30 mol of ethylene oxide to glycerol. Especially preferred are the C₁₀-C₁₆ fatty acid monoesters and diesters of adducts of 1 to 10 mol of ethylene oxide to glycerol. Especially preferred is the product known under the INCI name PEG-7 Glycerol Cocoate. Preferred compositions are characterized in that they include in addition an ethoxylated nonionic surfactant with an HLB value of 12 to 18.

Suitable oil and/or fat components can be preferably selected from mineral, natural, and synthetic oil components and/or fatty substances.

Triglycerides and mixtures of triglycerides are usually employed as natural (plant) oils. Preferred natural oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, cuckoo flower oil, thistle oil, macadamia nut oil, grape seed oil, amaranth seed oil, argan oil, bamboo oil, olive oil, wheat germ oil, pumpkin seed oil, mallow oil, hazelnut oil, safflower oil, canola oil, sasanqua oil, jojoba oil, rambutan oil, cocoa butter, and shea butter Preferred compositions according to the invention are characterized in that they include further a native oil, whereby the percentage by weight of the oil relative to the total weight of the composition is preferably 0.01 to 0.3% by weight, preferably from 0.05 to 0.1% by weight. Jojoba oil is an especially preferred native oil.

Mineral oils, paraffin oils, and isoparaffin oils, and synthetic hydrocarbons in particular are used as mineral oils. An example of a hydrocarbon which can be used is, for example, the commercially available product 1,3-di(2-ethylhexyl)cyclohexane (Cetiol® S).

A dialkyl ether can serve furthermore as an oil component. Dialkyl ethers which can be used are, in particular, di-n-alkyl ethers having a total of between 12 and 36 carbon atoms, particularly 12 to 24 carbon atoms, such as, for example, di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, as well as di-tert-butyl ether, di-isopentyl ether, di-3-ethyldecyl ether, tert-butyl n-octyl ether, isopentyl n-octyl ether, and 2-methylpentyl n-octyl ether. Especially preferred is the di-n-octyl ether, which is commercially available under the name Cetiol® OE.

Silicone compounds are suitable as synthetic oils.

Silicones bring about excellent conditioning properties on the hair. In particular, they bring about an improved combability of the hair in the wet and dry state and in many cases have a positive effect on the feel and softness of the hair.

It is therefore desirable to use silicones in cosmetic compositions. Suitable silicones (which differ from silicones a)) can be selected from:
(i) polyalkylsiloxanes, polyarylsiloxanes, polyalkylarylsiloxanes, which are volatile or nonvolatile, straight-chain, branched, or cyclic, crosslinked or non-crosslinked;
(ii) polysiloxanes which in their general structure include one or more organofunctional groups, which are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyloxyalkyl groups;
   h) amphoteric groups;
   i) bisulfite groups;
   j) hydroxyacylamino groups;
   k) carboxyl groups;
   l) sulfonic acid groups; and
   m) sulfate or thiosulfate groups;
(iii)linear polysiloxane (A)-polyoxyalkylene (B) block copolymers of the type (A-B)ₙ with n>3;
(iv) grafted silicone polymers having a non-silicone-containing, organic framework consisting of an organic main chain formed from organic monomers, containing no silicone, onto which at least one polysiloxane macromer has been grafted in the chain and optionally on at least one chain end;
(v) grafted silicone polymers having a polysiloxane framework, onto which non-silicone-containing, organic monomers have been grafted, which have a polysiloxane main chain onto which at least one organic macromer, containing no silicone, has been grafted in the chain and optionally on at least one of its ends;
(vi) or mixtures thereof.

The polydimethylsiloxanes are especially suitable as the synthetic oil. The group of polydimethylsiloxanes includes the linear as well as the nonlinear, preferably cyclic polydimethylsiloxanes. Polydimethylsiloxanes preferred according to the invention are selected from the linear polydimethylsiloxanes with a kinematic viscosity (25° C.) above 8000 cst, preferably above 10000 cst. The polydimethylsiloxane is preferably present in the cosmetic composition in dispersed form, whereby the droplet size of the dispersed polydimethylsiloxane is less than 10 μm, preferably less than 4 μm.

Fatty substances are understood to be fatty acids, fatty alcohols, and natural and synthetic waxes, which can be present both in solid form and in liquid form in aqueous dispersion.

Linear and/or branched, saturated and/or unsaturated fatty acids having 6 to 30 carbon atoms can be used as fatty acids. Fatty acids having 10 to 22 carbon atoms are preferred. Examples which can be cited include the isostearic acids, such as the commercial products Emersol® 871 and Emersol® 875, and isopalmitic acids such as the commercial product Edenor® IP 95, as well as all further fatty acids sold under the Edenor® trade names (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid, and erucic acid, and technical mixtures thereof.

The fatty acid cuts obtainable from coconut oil or palm oil are conventionally particularly preferred; as a rule the use of stearic acid is preferred in particular.

Saturated, mono- or polyunsaturated, branched or unbranched fatty alcohols having C₆ to C₃₀, preferably C₁₀ to C_{22,} and very especially preferably C₁₂ to C₂₂ carbon atoms can be used as fatty alcohols. For example, decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol, and behenyl alcohol, as well as the Guerbet alcohols thereof, can be used, whereby this list is intended to be of an exemplary and nonlimiting nature.

However, the fatty alcohols derive from preferably natural fatty acids, whereby it can conventionally be assumed that they are obtained from the esters of fatty acids by reduction. Fatty alcohol cuts which are produced by reduction of naturally occurring triglycerides such as beef tallow, palm oil, peanut oil, colza oil, cottonseed oil, soybean oil, sunflower oil, and linseed oil, or from fatty acid esters formed from the transesterification products thereof with corresponding alcohols and which thus represent a mixture of different fatty alcohols, can likewise be used according to the invention. Such substances can be purchased, for example, under the names Stenol®, e.g., Stenol® 1618, or Lanette®, e.g., Lanette® O, or Lorol®, e.g., Lorol® C8, Lorol® C14, Lorol® C18, Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. Wool wax alcohols can also be used, of course, according to the invention, as are available commercially, for example, under the names Corona®, White Swan®, Coronet®, or Fluilan®.

Solid paraffins or isoparaffins, carnauba waxes, beeswaxes, candelilla waxes, ozocerites, ceresin, sperm whale wax, sunflower wax, fruit waxes such as, for example, apple wax or citrus wax, PE or PP microwaxes can be used as natural or synthetic waxes. Such waxes are available, for example, via Kahl & Co., Trittau.

Other fatty substances are, for example,
ester oils. Ester oils are understood to be the esters of C₆-C₃₀ fatty acids with C₂-C₃₀ fatty alcohols. The monoesters of the fatty acids with alcohols having 2 to 24 C atoms are preferred. Examples of employed fatty acid components in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid, and erucic acid, and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol, and technical mixtures thereof. Isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V) are especially preferred.
dicarboxylic acid esters such as di-n-butyl adipate, di-(2-ethylhexyl)adipate, di-(2-ethylhexyl)succinate, and diisotridecyl acelate, as well as diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, and neopentyl glycol dicaprylate,
symmetric, asymmetric, or cyclic esters of carbonic acid with fatty alcohols,
glycerol carbonate or dicaprylyl carbonate (Cetiol® CC),
ethoxylated or non-ethoxylated mono-, di-, and tri-fatty acid esters of saturated and/or unsaturated linear and/or branched fatty acids with glycerol such as, for example, Monomuls® 90-O18, Monomuls® 90-L12, Cetiol° HE, or Cutina® MD.

Suitable antidandruff active agents can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts. Climbazole, zinc pyrithione, and piroctone olamine are preferred.

The further optional components, which may be used in the compositions according to the invention, include, for example,
vitamins, vitamin derivatives, and/or vitamin precursors,
plant extracts, and/or
humectants.

Suitable vitamins are understood preferably as the following vitamins, provitamins, and vitamin precursors, and derivatives thereof:
Vitamin A: the group of substances designated as vitamin A include retinol (Vitamin A₁) and 3,4-didehydroretinol (vitamin A₂). β-carotene is the retinol provitamin. Suitable vitamin A components are, for example, vitamin A acid and esters thereof, vitamin A aldehyde, and vitamin A alcohol and esters thereof such as the palmitate and the acetate.
Vitamin B: The vitamin B group or the vitamin B complex includes, inter alia,
   vitamin B₁ (thiamine)
   vitamin B₂ (riboflavin)
   vitamin B₃. The compounds nicotinic acid and nicotinic acid amide (niacinamide) are often included under this term.
   vitamin B₅ (pantothenic acid and panthenol). Panthenol is preferably used within the scope of this group. Usable panthenol derivatives are particularly the esters and ethers of panthenol and cationically derivatized panthenols. Individual representatives are, for example, panthenol triacetate, panthenol monoethyl ether, and the monoacetate thereof, as well as cationic panthenol derivatives.
   vitamin B₆ (pyridoxine as well as pyridoxamine and pyridoxal).
Vitamin C (ascorbic acid): the use in the form of the palmitic acid ester, glucosides, or phosphates can be preferred. The use in combination with tocopherols can likewise be preferred.
Vitamin E (tocopherols, especially a-tocopherol).
Vitamin F: The term “vitamin F” is conventionally understood to mean essential fatty acids, in particular linoleic acid, linolenic acid, and arachidonic acid.
Vitamin H: Vitamin H is the name for the compound (3aS,4S,6aR)-2-oxohexahydrothienol[3,4-d]-imidazole-4-valeric acid, although the trivial name biotin has become accepted in the meantime. The cosmetic compositions according to the invention can preferably include vitamins, provitamins, and vitamin precursors from the groups A, B, E, and H.

Preferred in particular are nicotinic acid amide, biotin, pantolactone, and/or panthenol.

Vitamins, vitamin derivatives, and/or vitamin precursors may be used in the cosmetic compositions (based on their total weight) preferably in an amount from 0.001 to 10% by weight, preferably 0.005 to 7.5% by weight, and especially 0.01 to 5% by weight.

Extracts that can be prepared from all parts of a plant are to be understood as suitable plant extracts. These extracts are typically produced by extraction of the entire plant. It can also be preferred in individual cases, however, to produce the extracts solely from the flowers and/or leaves of the plant. Suitable above all are the extracts from green tea, oak bark, stinging nettle, witch hazel, hops, chamomile, burdock, horsetail, whitethorn, lime blossom, lychee, almond, aloe vera, spruce needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger root, Echinacea purpurea, Olea europaea, Boerhavia diffilsa roots, Foeniculum vulgaris and Apium graveolens.

The extracts from green tea, stinging nettle, witch hazel, chamomile, aloe vera, ginseng, Echinacea purpurea, Olea europea, and/or Boerhavia diffusa roots are especially preferred for use in the compositions according to the invention.

Water, alcohols, and mixtures thereof can be used as extracting agents to produce the cited plant extracts. Of the alcohols, low alcohols such as ethanol and isopropanol, but in particular polyhydric alcohols such as ethylene glycol and propylene glycol are preferred, both as the sole extracting agent and in a mixture with water. Plant extracts based on water/propylene glycol in the ratio of 1:10 to 10:1 have proven to be particularly suitable.

The plant extracts can be used both in pure and diluted form. If they are used in diluted form they conventionally include about 2 to 80% by weight of active substance and as the solvent the extracting agent or mixture of extracting agents used to obtain them.

The plant extracts can be used in the cosmetic compositions (based on their total weight) preferably in an amount from 0.01 to 10% by weight, more preferably from 0.05 to 7.5% by weight, and especially from 0.1 to 5% by weight.

Suitable humectants or penetration aids and/or swelling agents that can be added to the cleaning agents according to the invention are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example, propylene glycol monoethyl ether, carbonates, hydrogen carbonates, diols and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, and 1,4-butanediol.

The humectants can be used in the compositions according to the invention, based on their total weight, preferably in amounts from 0.01 to 10% by weight, more preferably from 0.05 to 5% by weight, and especially from 0.1 to 3% by weight.

Other active substances, auxiliary substances, and additives, which can be used in the hair cleaning agents according to the invention, are, for example:
UV filters,
thickeners, such as gelatin or plant gum, for example, agar-agar, guar gum, alginates, xanthan gum, gum arabic, karaya gum, locust bean gum, flaxseed gums, dextrans, cellulose derivatives, e.g., methyl cellulose, hydroxyalkyl cellulose, and carboxymethyl cellulose, starch fractions and derivatives such as amylose, amylopectin, and dextrins, clays and phyllosilicates such as, e.g., bentonite, or fully synthetic hydrocolloids such as, e.g., polyvinyl alcohol, Ca, Mg, or Zn soaps,
structurants such as maleic acid and lactic acid,
dyes for coloring the agent,
substances for adjusting the pH, for example, α- and β-hydroxycarboxylic acids such as citric acid, lactic acid, malic acid, and glycolic acid,
active substance such as bisabolol,
complexing agents such as EDTA, NTA, β-alanine diacetic acid, and phosphonic acids,
ceramides. Ceramides are understood to be N-acylsphingosine (fatty acid amides of sphingosine) or synthetic analogs of such lipids (so-called pseudo-ceramides),
propellants such as propane-butane mixtures, N₂O, dimethyl ether, CO₂, and air,
antioxidants,
pearlescent agents such as EGDS or PEG-3 distearates,
preservatives such as, for example, sodium benzoate or salicylic acid,
viscosity regulators such as salts (NaCl).

The cosmetic compositions according to the invention preferably have pH values in the range from 2 to 7, preferably from 3 to 6, and especially from 4.5 to 5.5.

The frame formulations of some preferred compositions according to the invention can be obtained from the following tables (data are given in % by weight, based on the total weight of the compositions, unless otherwise stated).

| | Formula 1 | Formula 2 | Formula 3 | Formula 4 | Formula 5 |
|---|---|---|---|---|---|
| Anionic surfactant (A) | 6.0 to 15.0 | 6.0 to 15.0 | 7.0 to 13.0 | 8.0 to 11.0 | 8.0 to 11.0 |
| Amphoteric surfactant | 0 to 12 | 0 to 10 | 0 to 6.0 | 0 to 6.0 | 0 to 6.0 |
| (B) | | | | | |
| Sorbitan acid ester (C) | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 6 | Formula 7 | Formula 8 | Formula 9 | Formula 10 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 0 to 12 | 0 to 10 | 0 to 6.0 | 0 to 6.0 | 0 to 6.0 |
| Sorbitan acid ester (C) | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 11 | Formula 12 | Formula 13 | Formula 14 | Formula 15 |
|---|---|---|---|---|---|
| Anionic surfactant (A) | 6.0 to 15.0 | 6.0 to 15.0 | 7.0 to 13.0 | 8.0 to 11.0 | 8.0 to 11.0 |
| Amphoteric surfactant | 0 to 12 | 0 to 10 | 0 to 6.0 | 0 to 6.0 | 0 to 6.0 |
| (B) | | | | | |
| Sorbitan sesquioctanoate | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 16 | Formula 17 | Formula 18 | Formula 19 | Formula 20 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 0 to 12 | 0 to 10 | 0 to 6.0 | 0 to 6.0 | 0 to 6.0 |
| Sorbitan sesquioctanoate | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 21 | Formula 22 | Formula 23 | Formula 24 | Formula 25 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 0 to 12 | 0 to 10 | 0 to 6.0 | 0 to 6.0 | 0 to 6.0 |
| Sorbitan acid ester (C) | 0.1 to 3.0 | 0.2 to 2.0 | 0.2 to 2.0 | 0.25 to 1.0 | 0.25 to 1.0 |
| Polyquaternium-10 | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 26 | Formula 27 | Formula 28 | Formula 29 | Formula 30 |
|---|---|---|---|---|---|
| Anionic surfactant (A) | 4.0 to 7.0 | 4.0 to 7.0 | 5.0 to 6.0 | 5.0 to 6.0 | 5.0 to 6.0 |
| Amphoteric surfactant | 0 to 12 | 0 to 10 | 0 to 6.0 | 0 to 6.0 | 0 to 6.0 |
| (B) | | | | | |
| Sorbitan acid ester (C) | 0.1 to 3.0 | 0.2 to 2.0 | 0.2 to 2.0 | 0.25 to 1.0 | 0.25 to 1.0 |
| Polyquaternium-10 | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | | | | | |
|---|---|---|---|---|---|
| | 6.0 to 15.0 | 6.0 to 15.0 | 7.0 to 13.0 | 8.0 to 11.0 | 8.0 to 11.0 |
| Sodium lauryl sulfate | 4.0 to 7.0 | 4.0 to 7.0 | 5.0 to 6.0 | 5.0 to 6.0 | 5.0 to 6.0 |
| Cocamidopropyl betaine | 0 to 12 | 0 to 10 | 0 to 6.0 | 0 to 6.0 | 0 to 6.0 |
| Sorbitan sesquioctanoate | 0.1 to 3.0 | 0.2 to 2.0 | 0.2 to 2.0 | 0.25 to 1.0 | 0.25 to 1.0 |
| Polyquaternium-10 | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 36 | Formula 37 | Formula 38 | Formula 39 | Formula 40 |
|---|---|---|---|---|---|
| Anionic surfactant (A) | 6.0 to 15.0 | 6.0 to 15.0 | 7.0 to 13.0 | 8.0 to 11.0 | 8.0 to 11.0 |
| Amphoteric surfactant | 1.0 to 12 | 2.0 to 10 | 3.0 to 6.0 | 3.0 to 6.0 | 3.0 to 6.0 |
| (B) | | | | | |
| Sorbitan acid ester (C) | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 41 | Formula 42 | Formula 43 | Formula 44 | Formula 45 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 1.0 to 12 | 2.0 to 10 | 3.0 to 6.0 | 3.0 to 6.0 | 3.0 to 6.0 |
| Sorbitan acid ester (C) | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 46 | Formula 47 | Formula 48 | Formula 49 | Formula 50 |
|---|---|---|---|---|---|
| Anionic surfactant (A) | 6.0 to 15.0 | 6.0 to 15.0 | 7.0 to 13.0 | 8.0 to 11.0 | 8.0 to 11.0 |
| Amphoteric surfactant | 1.0 to 12 | 2.0 to 10 | 3.0 to 6.0 | 3.0 to 6.0 | 3.0 to 6.0 |
| (B) | | | | | |
| Sorbitan sesquioctanoate | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 51 | Formula 52 | Formula 53 | Formula 54 | Formula 55 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 1.0 to 12 | 2.0 to 10 | 3.0 to 6.0 | 3.0 to 6.0 | 3.0 to 6.0 |
| Sorbitan sesquioctanoate | 0.1 to 3.0 | 0.2 to 2.0 | 0.2 to 2.0 | 0.25 to 1.0 | 0.25 to 1.0 |
| Cat. polysaccharide (D) | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 56 | Formula 57 | Formula 58 | Formula 59 | Formula 60 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 1.0 to 12 | 2.0 to 10 | 3.0 to 6.0 | 3.0 to 6.0 | 3.0 to 6.0 |
| Sorbitan acid ester (C) | 0.1 to 3.0 | 0.2 to 2.0 | 0.2 to 2.0 | 0.25 to 1.0 | 0.25 to 1.0 |
| Polyquaternium-10 | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 61 | Formula 62 | Formula 63 | Formula 64 | Formula 65 |
|---|---|---|---|---|---|
| Anionic surfactant (A) | 6.0 to 15.0 | 6.0 to 15.0 | 7.0 to 13.0 | 8.0 to 11.0 | 8.0 to 11.0 |
| Amphoteric surfactant | 1.0 to 12 | 2.0 to 10 | 3.0 to 6.0 | 3.0 to 6.0 | 3.0 to 6.0 |
| (B) | | | | | |
| Sorbitan acid ester (C) | 0.1 to 3.0 | 0.2 to 2.0 | 0.2 to 2.0 | 0.25 to 1.0 | 0.25 to 1.0 |
| Polyquaternium-10 | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

| | Formula 66 | Formula 67 | Formula 68 | Formula 69 | Formula 70 |
|---|---|---|---|---|---|
| Sodium lauryl sulfate | 6.0 to 15.0 | 6.0 to 15.0 | 7.0 to 13.0 | 8.0 to 11.0 | 8.0 to 11.0 |
| Cocamidopropyl betaine | 1.0 to 12 | 2.0 to 10 | 3.0 to 6.0 | 3.0 to 6.0 | 3.0 to 6.0 |
| Sorbitan sesquioctanoate | 0.1 to 3.0 | 0.2 to 2.0 | 0.2 to 2.0 | 0.25 to 1.0 | 0.25 to 1.0 |
| Polyquaternium-10 | 0.05 to 2.0 | 0.1 to 2.0 | 0.1 to 2.0 | 0.1 to 1.0 | 0.2 to 0.5 |
| Misc. | to 100 | to 100 | to 100 | to 100 | to 100 |

As initially stated, the cleaning agents according to the invention have excellent properties during use on hair. Apart from cleaning, they give the hair treated therewith better optical and haptic properties. Treated dry hair (particularly damaged hair) has an improved feel and an improved combability. Treated wet hair (particularly damaged hair) has fewer or no knots and can be easily untangled. A further advantage of the cleaning agents according to the invention is that they promote the resistance of the hair surface to physical and/or chemical hair damage.

A further subject of the present application is a method for the treatment of hair, characterized in that a cleaning composition according to the invention is applied to wet hair and rinsed out with water after a treatment time of 30 seconds to 5 minutes.

A further subject of the invention is the use of a cosmetic composition according to the invention for improving the feel and combability of wet and dry hair and for improving the resisting power of the hair surface to physical and/or chemical hair damage.

### EXAMPLES

1) Preparation of a Shampoo According to the Invention:

| | I |
|---|---|
| Sodium laureth sulfate | 10.8 |
| Sorbitan sesquicaprylate | 0.9 |
| Coconut glycerides + 7.3 EO | 0.3 |
| PEG-12 dimethicone | 0.5 |
| Polyquaternium-10 | 0.7 |
| PEG-40 hydrogenated castor oil | 0.4 |
| Apricot kernel oil | 0.02 |
| Fragrance, preservative | q.s. |
| Citric acid (to pH 4.7 to 5.3) | 0.2 |
| NaCl (to a viscosity of 5000 to 8000 | 3.9 |
| [Brookfield 20 RM/20° C.] +/− 1000 mPas) | |
| Water | To 100 |

2) Evaluation of the Shampoos:

2.1 Evaluation of Wet Combability

a) Hair strands/measuring device: Hair strands from the company Kerling International (Backnang, Germany) were used: European natural hair 7/0; lot #04/2010; N74; length: 12 cm; weight: 1+/−0.05 g. Measuring device: Hercules Sagemann (Hamburg, Germany) and hard rubber comb, fine tine.

b) Treatment of the hair strands and measurement: The hairs strands were first damaged in that they were bleached twice and then stored at 25° C. and 25% humidity for 48 hours. 0.25 g of the particular shampoo was applied per gram of hair strand. After 2 minutes, the hair strands were rinsed with water (32° C., 0.5 L/min) for 18 seconds, while they were combed automatically. This procedure was repeated once. Before the measurement, each hair strand was moistened with water for 2 seconds. After each hair strand was precombed (3 strokes), each hair strand was combed 10 times, whereby the work expended thereby was measured (in this case, the hair strands were slowly turned). The expended work was compared with the decrease in wet combability. (Here, the following applies: the higher the numerical value for the decrease in wet combability, the lower the expended work to pull the comb through the hair strands). The wet combability was measured using 12 hair strands for each shampoo.

2.2 Evaluation of the Detangling and Hair Feel in the Hair Strand Test by Experts

In a second test, in eight test series three hair strands in each case were evaluated by two independent experts (a, b) in regard to the disentangleability (in the wet state) and the feel (softness, suppleness) of the hair strands in the dry state. The hair strands used were machine wefts from European natural hair; remis; double drawn; color 7/0; 3 cm wide, 25 cm total length; about 2 g total weight (Kerling Int. Haarfabrik GmbH). The strands were first damaged in that they were treated with a hair dye (80 g of “Igora Royal color & care developer,” which was combined with 20 g of “Igora blue dust-free bleach”), and rinsed with water after 30 minutes for 20 seconds. Next, the strands were washed in each case with 1 mL of a 10% sodium lauryl ether sulfate solution, rinsed with water for 20 seconds, and wrung out. 1 g of the particular test shampoos was filled in each case into a petri dish. Next, the predamaged hair strands were briefly immersed into the particular test shampoos and rinsed out with water. The strands were evaluated by the two experts in the dry and wet state.

The hair strands treated with the composition according to the invention were notable for a better-than-expected wet combability, good disentangleability, and great suppleness.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cleaning cosmetic composition, which includes, in a cosmetic carrier,
a) at least one anionic surfactant (A),
b) optionally an amphoteric surfactant (B),
c) at least one sorbitan acid ester (C),
d) at least one cationic polysaccharide (D).

2. The composition according to claim 1, characterized in that the at least one anionic surfactant (A) is an alkyl sulfate or alkyl polyglycol ether sulfate of the formula R—O—(CH₂—CH₂O)ₙ—O—SO₃X, in which R is a straight-chain or branched, saturated or mono- or polyunsaturated alkyl or alkenyl group having 8 to 24 carbon atoms, n is 0 to 12, and X is an alkali metal or alkaline earth metal or triethanolamine.

3. The composition according to claim 1, characterized in that the at least one amphoteric surfactant (B) hsa the general formula (iii)

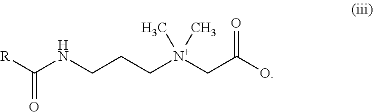

4. The composition according to claim 1 characterized in that the percentage of the anionic surfactant (A) and amphoteric surfactant (B) in the composition, based on the total weight of the composition, is 6.0 to 15% by weight.

5. The composition according to claim 1, characterized in that sorbitan sesquioctanoate is the sorbitan acid ester (C).

6. The composition according to claim 1, characterized in that the percentage of the sorbitan acid ester (C) relative to the total weight of the composition is 0.1 to 3.0% by weight.

7. The composition according to claim 1, characterized in that the at least one cationic polysaccharide (D) is a cationic guar derivative.

8. The composition according to claim 1, characterized in that the percentage of the cationic polysaccharide (D) relative to total weight of the composition is 0.01 to 1.0% by weight.

9. The composition according to claim 1, further including a native oil (E), wherein the percentage of the oil (E) relative to the total weight of the composition is 0.01 to 0.3% by weight.

10. A method for hair treatment, including applying a cosmetic cleaning composition according to claim 1 to wet hair, and rinsing the composition out with water after a treatment time of 30 seconds to 5 minutes.
